(19) United States
(12) Patent Application Publication
(10) Pub. No.: US 2009/0325270 A1
(43) Pub. Date: Dec. 31, 2009

(54) USE OF 2-HYDROXY-5-OXOPROLINE IN CONJUNCTION WITH ALGAE

(76) Inventors: Bertrand Vick, Emeryville, CA (US); Daniel Fleischer, Oakland, CA (US)

Correspondence Address: CARR & FERRELL LLP, 2200 GENG ROAD, PALO ALTO, CA, 94303, US

(21) Appl. No.: 12/492,077

(22) Filed: Jun. 25, 2009

Related U.S. Application Data
(60) Provisional application No. 61/133,168, filed on Jun. 25, 2008.

(51) Int. Cl. C12N 1/12; C12M 1/00

(52) U.S. Cl. 435/257.1; 435/289.1

(57) ABSTRACT

Provided herein are exemplary methods for the use of 2-hydroxy-5-oxoproline in conjunction with algae. One exemplary method includes applying an effective amount of 2-hydroxy-5-oxoproline to algae in an aqueous environment to accelerate creation of a high-cell density of the algae. The effective amount of the 2-hydroxy-5-oxoproline may be approximately 0.1 grams per liter of the aqueous environment, or up to approximately 0.1 grams per liter of the aqueous environment. The effective amount of the 2-hydroxy-5-oxoproline may be applied to the aqueous environment at or near a same time, or applied to the aqueous environment over a period of time. Exemplary algae cultivation systems are also provided herein. One exemplary system includes an aqueous environment having a pale-green mutant Nannochloropsis, and an effective amount of 2-hydroxy-5-oxoproline to accelerate creation of a high-cell density of the pale-green mutant Nannochloropsis.

### CROSS-REFERENCE TO RELATED APPLICATIONS

The present application claims the benefit and priority of U.S. Provisional Patent Application Ser. No. 61/133,168 filed on Jun. 25, 2008 titled “The Use of 2-Hydroxy-5-Oxoproline in Conjunction with Nannochloropsis Locked in a High-Light Acclimated State,” which is hereby incorporated by reference.

### BACKGROUND OF THE INVENTION

1. Field of the Invention

This invention relates to the cultivation of algal cells, and more particularly to the use of 2-hydroxy-5-oxoproline in conjunction with algae.

2. Description of Related Art

Open raceway ponds (and other open-air pond designs) provide a relatively inexpensive and scalable solution for culturing photosynthetic micro-organisms. Spirulina (a cyanobacterium) and Dunaliella salina (a microalga), for example, may be cultivated in an open pond architecture over tens, hundreds or even thousands of acres. Many companies take advantage of open ponds for the commercial production of microbial biomass for many different purposes, including energy, nutraceuticals and animal feed. Nevertheless, the large-scale cultivation of organisms in open ponds for producing fuel feedstock presents formidable challenges. Many of the challenges pertain directly to the biomass productivity of the organism(s) cultivated.

### SUMMARY OF THE INVENTION

Provided herein are exemplary methods for the use of 2-hydroxy-5-oxoproline in conjunction with algae. One exemplary method includes applying an effective amount of 2-hydroxy-5-oxoproline to algae in an aqueous environment to accelerate creation of a high-cell density of the algae. The algae may be a wild-type Nannochloropsis, a pale-green mutant Nannochloropsis, a wild-type algae, a pale-green algae established by manipulation of growth conditions of the aqueous environment, or algae treated with a chemical or a genetic method to reduce an amount of chlorophyll in the algae. The effective amount of the 2-hydroxy-5-oxoproline may be approximately 0.1 grams per liter of the aqueous environment, or up to approximately 0.1 grams per liter of the aqueous environment. The effective amount of the 2-hydroxy-5-oxoproline may be applied to the aqueous environment at or near a same time, or applied to the aqueous environment over a period of time.

Exemplary algae cultivation systems are also provided herein. One exemplary system includes an aqueous environment having a pale-green mutant Nannochloropsis, and an effective amount of 2-hydroxy-5-oxoproline to accelerate creation of a high-cell density of the pale-green mutant Nannochloropsis. The aqueous environment may include seawater, fresh water, or a mixture of seawater and fresh water. The algae cultivation system may be in a photobioreactor, a pond, or a vessel.

### BRIEF DESCRIPTION OF THE DRAWINGS

FIG. 1 illustrates an exemplary method for generating fuel feedstock by the cultivation of a pale-green mutant Nannochloropsis in an effective amount of 2-hydroxy-5-oxoproline.

FIG. 2 illustrates an exemplary algae cultivation system for generating fuel feedstock by the cultivation of a pale-green mutant Nannochloropsis in an effective amount of 2-hydroxy-5-oxoproline.

FIG. 3 is a graph showing exemplary algae growth in an aqueous environment under varying conditions, including treatment with an effective amount of 2-hydroxy-5-oxoproline, as described in connection with Example One.

### DETAILED DESCRIPTION

Provided herein are exemplary methods and systems for the use of 2-hydroxy-5-oxoproline in conjunction with algae. One exemplary embodiment includes the cultivation of algae or pale green algae in an effective amount of 2-hydroxy-5-oxoproline. Another exemplary embodiment includes the cultivation of wild-type Nannochloropsis in an effective amount of 2-hydroxy-5-oxoproline. A further exemplary embodiment includes the cultivation of pale-green mutant Nannochloropsis in an effective amount of 2-hydroxy-5-oxoproline.

According to various exemplary embodiments, algae, such as Nannochloropsis, is about 3 to 5 micrometers in size and may be cultivated in an aqueous environment. In low-light conditions, various forms of algae, such as wild-type Nannochloropsis, acclimates in part by increasing the amount of chlorophyll in the cell and turning a dark green. In high-light conditions, the algae, including wild-type Nannochloropsis, acclimates by reducing its chlorophyll content and turning a pale-green. According to a further exemplary embodiment, Nannochloropsis may be locked in the high-light acclimated state through mutagenesis to produce a pale-green mutant Nannochloropsis. Mutant Nannochloropsis, in general, may be Nannochloropsis that has been treated with chemicals or molecular genetic methods to reduce the amount of chlorophyll in the cell. Various forms of pale green algae, including Nannochloropsis, encompasses cells that have reduced light harvesting antennae and/or cells that are high-light acclimated. Further, algae, including pale green Nannochloropsis, may be established by manipulating growth conditions of an aqueous environment. In addition, a plant growth regulator, such as 2-hydroxy-5-oxoproline, may be used to increase the growth rate of algae toward high-cell density.

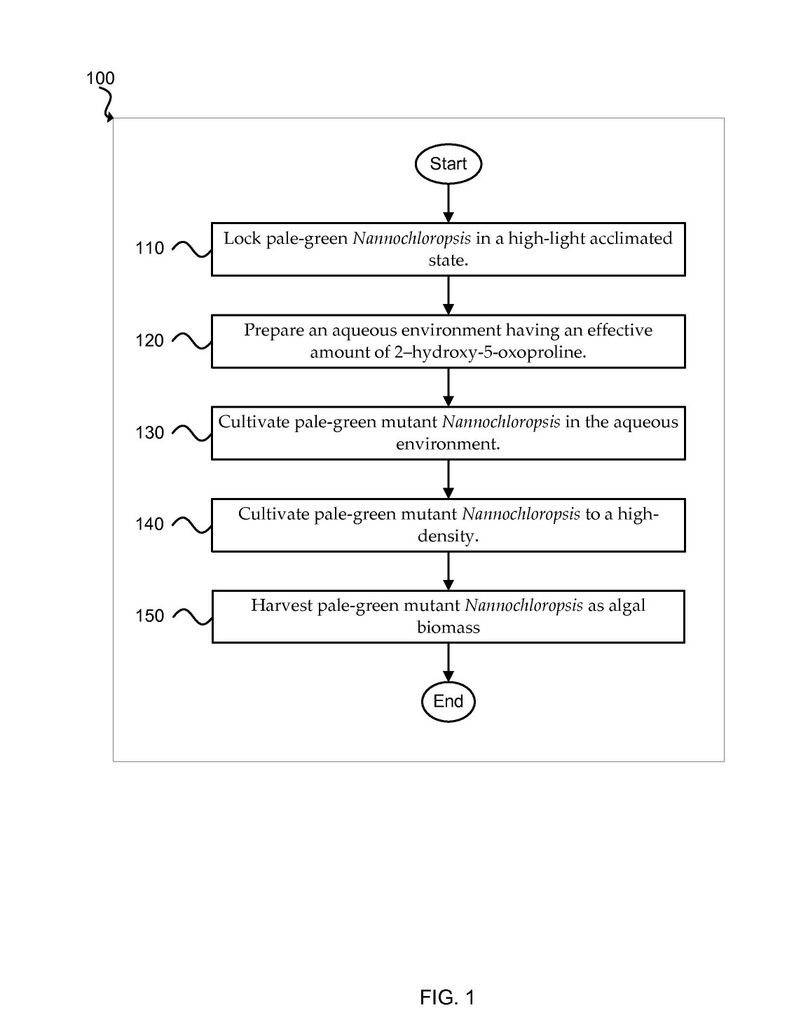

FIG. 1 illustrates one exemplary method 100 for generating fuel feedstock by the cultivation of pale-green mutant Nannochloropsis in an effective amount of 2-hydroxy-5-oxoproline.

At step 110, Nannochloropsis may be locked in a mutated pale-green state of high-light acclimation. Locking the pale-green Nannochloropsis in the high-light acclimated state results in an algal cell that does not increase its chlorophyll content in low-light conditions. Even in dense algae cultures, the pale-green mutant Nannochloropsis retains less chlorophyll and remains pale-green. In addition, the pale-green mutant Nannochloropsis grows to a much higher density than observed in a wild-type Nannochloropsis culture. Consequently, the mutant Nannochloropsis has higher biomass productivity at a high-cell density, and generally performs better in mass culture.

At step 120, an aqueous environment is prepared with an effective amount of 2-hydroxy-5-oxoproline. In one embodiment, the effective amount may be approximately 0.1 grams per liter. In other embodiments, the effective amount may be up to approximately 0.1 grams per liter. According to further embodiments, the effective amount of 2-hydroxy-5-oxoproline may be added to the aqueous environment all at once, or it may be added to the aqueous environment in smaller amounts over time. Additionally, the effective amount of 2-hydroxy-5-oxoproline may vary from less than approximately 0.1 grams per liter to greater than approximately 0.9 grams per liter.

In various embodiments, the 2-hydroxy-5-oxoproline may be synthesized from the reaction of glutamine with Fremy's salt. According to one embodiment, 5 grams of glutamine is reacted with Fremy's salt in a volume of 500 milliliters of buffer. Ten milliliters of the solution may be added to an algal culture. In a further embodiment, 10 grams of glutamine may be converted to 2-hydroxy-5-oxoproline to yield a total of 0.1 g of 2-hydroxy-5-oxoproline for addition to an algal culture.

At step 130, the pale-green mutant Nannochloropsis may be cultivated in an aqueous environment having an effective amount of 2-hydroxy-5-oxoproline. The effective amount of 2-hydroxy-5-oxoproline may increase the growth rate of the pale-green mutant Nannochloropsis. According to one embodiment, a pale-green mutant Nannochloropsis cultivated with an effective amount of 2-hydroxy-5-oxoproline may grow fifty to sixty percent faster (as measured by absorbance at 750 nm), than a pale-green mutant Nannochloropsis cultivated without an effective amount of 2-hydroxy-5-oxoproline.

According to various embodiments, the pale-green mutant Nannochloropsis may require light (natural or artificially supplied) for growth, as well as nutrients. Other parameters such as pH should be within acceptable ranges. The basic elements typically required for pale-green mutant Nannochloropsis growth may include carbon, oxygen, hydrogen, nitrogen, sulfur, phosphorous, potassium, magnesium, iron and traces of several other elements.

The required nutrients for pale-green mutant Nannochloropsis growth may be contained in the water, supplied subsequently in dilution waters, or supplied independently of the dilution waters. The amount of nutrients needed to yield a prescribed pale-green mutant Nannochloropsis density may be determined by the cell quota for that nutrient. That is, by the per cent of the algal dry mass that is comprised of the element contained in the nutrient. The inverse of the cell quota is called the algae growth potential for that nutrient or element. For instance, if the desired final density is 1 gram/liter and the pale-green mutant Nannochloropsis under consideration contains ten percent (10%) nitrogen in its biomass (i.e., a cell quota of 0.1), then the initial concentration of the atomic nitrogen in the culture should be at least 0.1 gram/liter. The same calculation may be performed for all nutrients to establish their initial concentration in the culture.

In various embodiments, the time-averaged light intensity to which pale-green mutant Nannochloropsis may be exposed may be adjusted by changes in the mixing intensity and/or in the optical depth of the pond. The optical depth in open ponds may be the depth of the pond. In open ponds, the temperature may be controlled by adjusting culture depth.

At step 140, the pale-green mutant Nannochloropsis reaches a high-cell density. The high-cell density may be about 300 mg algal biomass per liter.

At step 150, the pale-green mutant Nannochloropsis may be harvested as algal biomass.

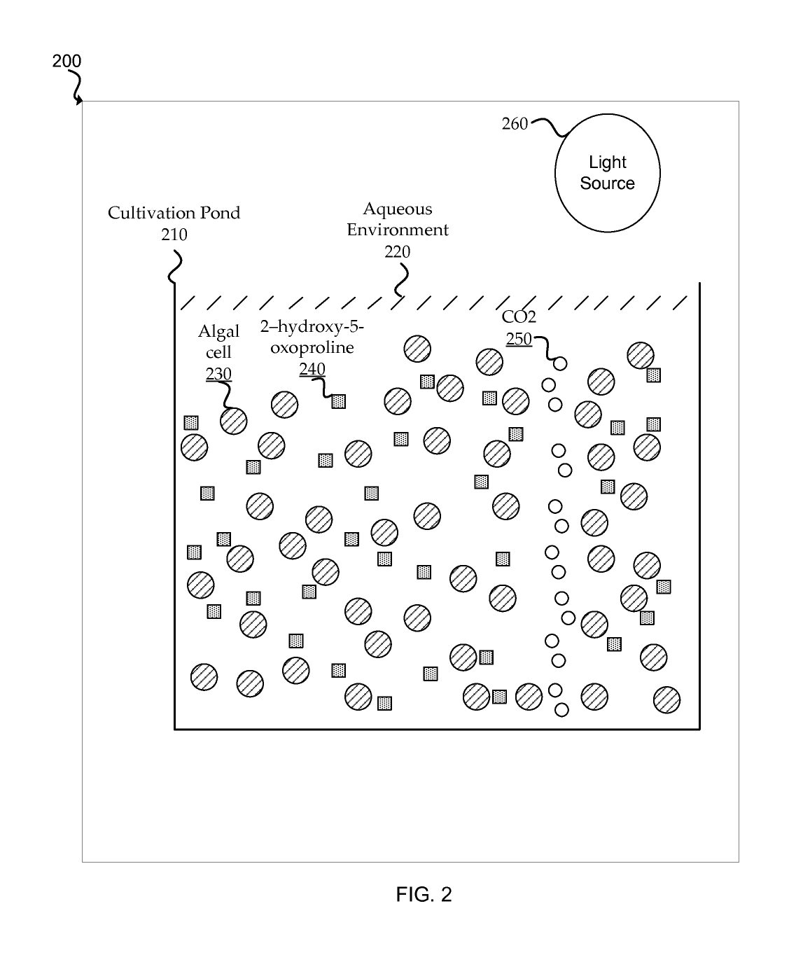

FIG. 2 illustrates an exemplary algae cultivation system 200 for generating fuel feedstock by the cultivation of a pale-green mutant Nannochloropsis in an effective amount of 2-hydroxy-5-oxoproline. The exemplary apparatus 200 may comprise a cultivation pond 210, an aqueous environment 220, a pale-green mutant Nannochloropsis 230, an effective amount of 2-hydroxy-5-oxoproline 240, an inorganic carbon 250, and/or a light source 260.

The cultivation pond 210, according to one embodiment, may be an open-air pond, lake or other body of water. In other embodiments, the cultivation pond 210 may be an open-air container, such as a pool or dish. Other embodiments may be partially or wholly sealed, such as an enclosed pool, a flask, and/or a bioreactor.

An aqueous environment 220 may be within the cultivation pond 210. In various embodiments, the aqueous environment 220 may partially fill the cultivation pond 210. In some embodiments, the aqueous environment 220 may wholly fill the cultivation pond 210.

A pale-green mutant Nannochloropsis 230 may be cultivated within the aqueous environment 220. In various embodiments, the pale-green mutant Nannochloropsis 230 may be locked in a high-light acclimated state.

An effective amount of 2-hydroxy-5-oxoproline 240 may be within the aqueous environment 220. In various embodiments, the effective amount may be approximately 0.1 grams of 2-hydroxy-5-oxoproline 240 per liter of aqueous environment 220. In other embodiments, the effective amount may be up to approximately 0.1 grams per liter.

An inorganic carbon 250 may be bubbled, sparged or otherwise distributed within the aqueous environment 220. In various embodiments, the inorganic carbon 250 may be carbon dioxide in pure form. In some embodiments, the inorganic carbon 250 may be a mixture of other gases. According to at least one embodiment, the inorganic carbon 250 may be bicarbonate.

A light source 260 may illuminate the cultivation pond 210 for cultivating the pale-green mutant Nannochloropsis 230 to reach a high-cell density.

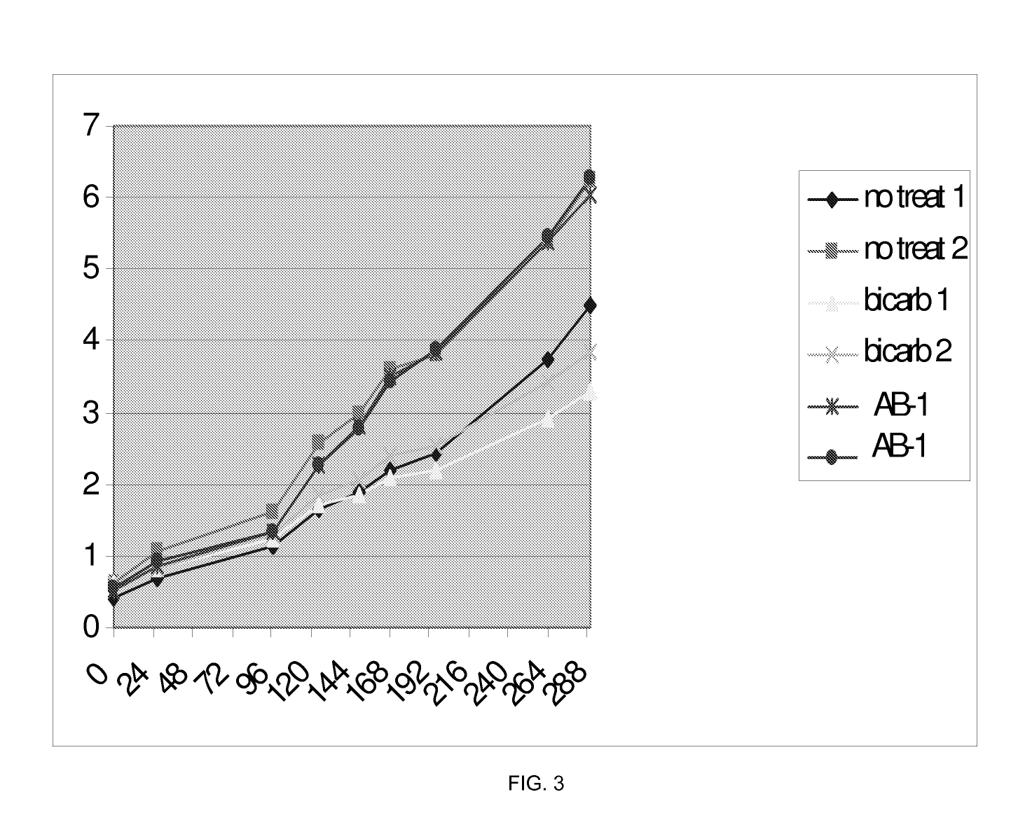

FIG. 3 is a graph showing exemplary algae growth in an aqueous environment under varying conditions, including treatment with an effective amount of 2-hydroxy-5-oxoproline, as described in connection with Example One.

### EXAMPLE ONE

1. Light intensity was 600 micro-Einsteins.

2. Temperature was held constant at 25 C.

3. Cultures were inoculated to the same extent (as determined by optical density at 700 nm, O.D. 750).

4. The bicarb controls were used because the AB1 chemical, which is the 2-hydroxy-5-oxoproline compound, was dissolved in a bicarbonate buffer. This control was to make sure that differences in growth were not due to the presence of bicarbonate. The bicarb controls contain the same concentration of bicarbonate as the AB1 flasks.

5. The cultures were grown on urea as the nitrogen-source.

As shown in FIG. 3, no treat 1, bicarb 1 and bicarb 2 represent controls whereby the same number of cells as in AB-1 were inoculated into 800 mls of algae-growth media and allowed to grow, without dilution, for approximately 300 hours under constant illumination. The bicarb controls were inoculated with 10 mls of 1.3 M bicarbonate solution (pH 9.5). The AB-1 treated cultures had approximately 0.1 g of 2-hydroxy-5-oxoproline added with the algal inoculum. No treat 2 had approximately 50% more cells inoculated as no treat 1, bicarb 1 and 2 and the AB-1 cultures. Comparing no treat 2 with the AB-1 treated cultures, AB-1 treated cultures grew as if they had been inoculated with more cells than had actually been added. The difference in slope between the AB-1 cultures and the no-treat 1 and bicarb controls was approximately 50%-60%. Therefore, by feeding 2-hydroxy-5-oxoproline to pale-green mutant Nannochloropsis, a greater growth rate could be achieved, which would allow high cell density cultures to be produced quicker, so the advantages of the pale-green phenotype over the wild-type become apparent.

### EXAMPLE TWO (PROPHETIC)

A Nannochloropsis cultivar is mutagenized by exposure to ultraviolet radiation of an intensity and duration sufficient to kill less than 100% of the cells. The surviving cells are plated on agar media, with a cell density low enough to enable visual screening of colonies by color. Pale green colonies are selected and isolated. The isolated pale green mutants are cultivated in growth conditions similar to those found in open pond cultivation, to identify one that has enhanced growth characteristics at high cell density. This strain (the pale green mutant) is then inoculated in the presence of 2-hydroxy-5-oxoproline at a concentration of 0.1 grams per liter of culture medium. The pale green mutant Nannochloropsis reaches a high cell density in a relatively short period of time in the presence of the 2-hydroxy-5-oxoproline.

### EXAMPLE THREE (PROPHETIC)

A wild-type Nannochloropsis cultivar is plated on agar media. The wild-type Nannochloropsis cultivar is cultivated in growth conditions similar to those found in open pond cultivation. The wild-type Nannochloropsis cultivar is then inoculated in the presence of 2-hydroxy-5-oxoproline at a concentration of approximately 0.1 grams per liter of culture medium. The treated wild-type Nannochloropsis cultivar reaches a high cell density faster than an untreated wild-type Nannochloropsis cultivar.

What is claimed is:

1. A method for generating fuel feedstock, the method comprising:
applying an effective amount of 2-hydroxy-5-oxoproline to algae in an aqueous environment to accelerate creation of a high-cell density of the algae.

2. The method of claim 1, wherein the algae is wild-type Nannochloropsis.

3. The method of claim 1, wherein the algae is pale-green mutant Nannochloropsis.

4. The method of claim 1, wherein the algae is wild-type algae.

5. The method of claim 1, wherein the algae is pale-green algae established by manipulation of growth conditions of the aqueous environment.

6. The method of claim 1, the method further comprising treating the algae with a chemical or a genetic method to reduce an amount of chlorophyll in the algae.

7. The method of claim 1, wherein the algae have reduced light harvesting antennae.

8. The method of claim 1, wherein the algae is acclimated to high-light intensity.

9. The method of claim 1, wherein the effective amount of the 2-hydroxy-5-oxoproline is approximately 0.1 grams per liter of the aqueous environment.

10. The method of claim 1, wherein the effective amount of the 2-hydroxy-5-oxoproline is up to approximately 0.1 grams per liter of the aqueous environment.

11. The method of claim 1, wherein the effective amount of the 2-hydroxy-5-oxoproline is applied to the aqueous environment at or near a same time.

12. The method of claim 1, wherein the effective amount of the 2-hydroxy-5-oxoproline is applied to the aqueous environment over a period of time.

13. The method of claim 1, wherein the effective amount of the 2-hydroxy-5-oxoproline is in a range of approximately 0.1 grams per liter of the aqueous environment to approximately 0.9 grams per liter of the aqueous environment.

14. An algae cultivation system for generating fuel feedstock, the algae cultivation system comprising:
an aqueous environment having a pale-green mutant Nannochloropsis; and
an effective amount of 2-hydroxy-5-oxoproline to accelerate creation of a high-cell density of the pale-green mutant Nannochloropsis.

15. The algae cultivation system of claim 14, wherein the aqueous environment includes seawater.

16. The algae cultivation system of claim 14, wherein the aqueous environment includes fresh water.

17. The algae cultivation system of claim 14, wherein the aqueous environment includes a mixture of seawater and fresh water.

18. The algae cultivation system of claim 14, wherein the algae cultivation system is in a photobioreactor.

19. The algae cultivation system of claim 14, wherein the algae cultivation system is in a pond.

20. The algae cultivation system of claim 14, wherein the algae cultivation system is in a vessel.
